(19) United States
(12) Patent Application Publication
(10) Pub. No.: US 2016/0271051 A1
(43) Pub. Date: Sep. 22, 2016

(54) COMPOSITION FOR PRODUCING COSMETICS USING PLANT MATERIAL EXTRACT AND FERMENTATION MATERIAL THEREOF AND METHOD FOR PRODUCING SAME

(71) Applicant: BST INC., Gyeonggi-do (KR)

(72) Inventors: Ho LEE, Seoul (KR); Chul KIM, Gyeonggi-do (KR); Seok Joon LEE, Gyeonggi-do (KR); Won Kyun PARK, Seoul (KR); Su Kyun KIM, Gangwon-do (KR)

(21) Appl. No.: 14/778,082

(22) PCT Filed: Oct. 2, 2013
(86) PCT No.: PCT/KR2013/008822

§ 371 Date: Sep. 18, 2015

(30) Foreign Application Priority Data
Mar. 19, 2013 (KR) 10-2013-0028937
Aug. 26, 2013 (KR) 10-2013-0101044
Sep. 30, 2013 (KR) 10-2013-0116601

(51) Int. Cl. A61K 8/97; C12P 1/04; A61Q 17/04; A61Q 19/00; A61Q 5/00

(52) U.S. Cl. CPC A61K 8/97; A61Q 19/00; A61Q 5/006; A61Q 17/04; C12P 1/04; A61K 2800/85

(57) ABSTRACT

The present invention relates to a composition for producing cosmetics using a plant material extract or a fermentation material thereof and a method for producing same and, more particularly, to a composition using a plant material extract or a fermentation material thereof, comprising an active ingredient extracted from a plant material or consisting of a fermentation material of the active ingredient and being applicable as an ingredient of functional cosmetics, and a method for producing same. The composition according to the present invention comprises at least one selected from the group consisting of Taraxacum platycarpum, Centella asiatica, Ecklonia stolonifera, Bambuseae, Petasites japonicus, Chamaecyparis obtusa, Cactaceae and Aloe and comprises an extract which has been extracted from the at least one by means of hot-water extraction, ethanol extraction, microwave extraction or supercritical extraction, or a fermentation ingredient of the extract, wherein the fermentation ingredient has been fermented using Lactobacillus.

### FIELD OF THE INVENTION

The present invention relates to a composition for producing cosmetics using a plant material extract or a fermentation material thereof and a method for producing same and, more particularly, to a composition using a plant material extract or a fermentation material thereof, comprising an active ingredient extracted from a plant material or consisting of a fermentation material of the active ingredient and being applicable as an ingredient of functional cosmetics, and a method for producing same.

### BACKGROUND ART

Functional cosmetics may include ingredients having the effects of treating (or curing) diverse forms of illnesses (or diseases), and, for example, such functional cosmetics may include materials enhancing blood flow, treating acne, generating carboxy, or such functional cosmetics may include ingredients having effects of treating pityriasis (or dandruff) and tinea pedis (or Athlete's foot). Additionally, functional cosmetics may include ingredients for blocking ultra-violet (UV) rays. Diverse forms of compositions for such functional effects are disclosed.

The Korean Patent Publication No. 2011-0019411 discloses a set of starter for producing long-term fermented kimchi for enhancing blood flow, which includes Lactobacillus paraplantarum, Weissella cibaria, Leuconostoc mesenteroide, Lactococcus lactis, and Saccharomyces Cerevisiae. Additionally, the Korean Patent Publication No. 2009-0127523 discloses a composition for acne treatment. And, the Korean Patent Publication No. 2001-0031864 discloses a viscous cosmetics composition containing carbon dioxide.

On the other hand, the Korean Patent Publication No. 2005-0117409 discloses a anti-dandruff hair cosmetics composition, which uses a Fritillaria verticillata extract as its active ingredient, wherein the extract is extracted by using an extraction solvent being selected from a group consisting of water, methanol, ethanol, propanol, butonal, glycerol, propylene glycol, 1,3-Butylene Glycol. And, the Korean Patent Publication No. 2007-0017451 discloses a tinea pedis treatment, which is configured of supaclene; at least one selected from citral, carvacrol, and timol; at least one selected from sodium benzoate, allantoin, and Sodium propionate; and at least one selected from dietary fiber and soluble collagen.

As a prior art related to blocking UV rays, there is the Korean Patent Publication No. 2013-0087650. This prior art discloses a sunblock composition configured of a hydrophobic liquid polymer medium for at least one sunblock activator selected from a group consisting of a phenolic acid compound, a flavonoid compound, and a derivative of such compounds, and for an impregnation of such sunblock activator. And, the Korean Patent Publication No. 2008-0072997 discloses a soluble UV ray blocking ingredient, which includes cyclodextrin and 2,4-bis-(4-(2-ethyl-hexyloxy)-2-hydrodiphenyl-6-(4-methoxyphenyl)-(1,3,5)-triazinol, and wherein the cyclodextrin and the sunblock absorbent has a mole ratio of 3:1.

The prior art discloses effects of specific infections or diseases (or illnesses) that are caused in the human body. However, the prior art is disadvantageous in that it does not include any ingredients based on plant materials, which correspond to natural ingredients.

The present invention has the following objects in order to resolve the problems shown in the prior art.

### DETAILED DESCRIPTION OF THE INVENTION

### Technical Objects

An object of the present invention is to provide a composition for producing cosmetics using a plant material extract or a fermentation material thereof and a method for producing the same being based on plant materials and having effects on diverse forms of infection or illnesses occurring in the human body.

### Technical Solutions

According to an adequate exemplary embodiment of the present invention, a composition for producing functional cosmetics includes at least one selected from the group consisting of Taraxacum platycarpum, Centella asiatica, Ecklonia stolonifera, Bambuseae, Petasites japonicus, Chamaecyparis obtusa, Cactaceae and Aloe and comprises an extract which has been extracted from the at least one by means of hot-water extraction, ethanol extraction, microwave extraction or supercritical extraction, or a fermentation ingredient of the extract, wherein the fermentation ingredient has been fermented using Lactobacillus.

According to another adequate exemplary embodiment of the present invention, the cosmetics may correspond to functional cosmetics for enhancing blood flow, treating acne, generating carboxy, treating pityriasis (or dandruff), treating tinea pedis (or Athlete's foot), blocking UV rays, or relieving epidermal infections.

According to yet another adequate exemplary embodiment of the present invention, the extract or fermentation ingredient includes at least one selected from a group consisting of coumarinic acid, cinnamic acid, ferulic acid, aciatic acid, and asiaticoside.

According to yet another adequate exemplary embodiment of the present invention, a method for producing a composition for producing function cosmetics using a plant material extract or a fermentation material thereof includes the steps of preparing at least one plant material selected from a group consisting of Taraxacum platycarpum, Centella asiatica, Ecklonia stolonifera, Bambuseae, Petasites japonicus, Chamaecyparis obtusa, Cactaceae and Aloe; forming an extract by processing the at least one plant material by means of hot-water extraction, ethanol extraction, microwave extraction or supercritical extraction; and forming a concentrated solution of the extract.

According to a further adequate exemplary embodiment of the present invention, the method further includes a step of fermenting the extract by using Lactobacillus.

### Effects of the Invention

Being added as an ingredient for functional cosmetics the composition according to the present invention may have the advantageous effects of enhancing blood flow (or blood circulation), acne treatment, generating carboxy, pityriasis (or dandruff), tinea pedis (or Athlete's foot), or blocking ultra-violet (UV) rays (sunblock or sunscreen). Additionally, the composition according to the present invention is advantageous in that it may be added to cosmetic products or functional medications without requiring any separate treatment processes. Furthermore, the composition according to the present invention is advantageous in that, since the composition ensures stability in diverse environmental conditions, the effects of the composition may be maintained during a predetermined period of time, and, since the composition shows no side effects, the composition may also be used as a base material for cosmetics compositions having diverse functions.

### BRIEF DESCRIPTION OF THE DRAWINGS

FIG. 1 illustrates a general view showing a process of producing a composition according to one exemplary embodiment of the present invention.

FIG. 2a and FIG. 2b respectively show exemplary embodiments of effects on pityriasis (or dandruff) and tinea pedis (or Athlete's Foot) by using the composition according to the present invention.

FIG. 3 illustrates an exemplary embodiment of pharmaceutical effects of the composition according to the present invention.

### BEST MODE FOR CARRYING OUT THE PRESENT INVENTION

Although the present invention will hereinafter be described in detail with reference to the exemplary embodiments presented in the accompanying drawings, the description is merely provided for a clear understanding on the present invention, and, therefore, the present invention will not be limited only to this. In the following description, since elements having the same reference numerals in different drawings have similar functions, unless required for the understanding of the present invention, the corresponding elements will not be repeatedly described, and, although the disclosed elements may only be briefly described, or although the description of the same may be omitted, it shall not be understood that the corresponding elements are excluded from the exemplary embodiments of the present invention.

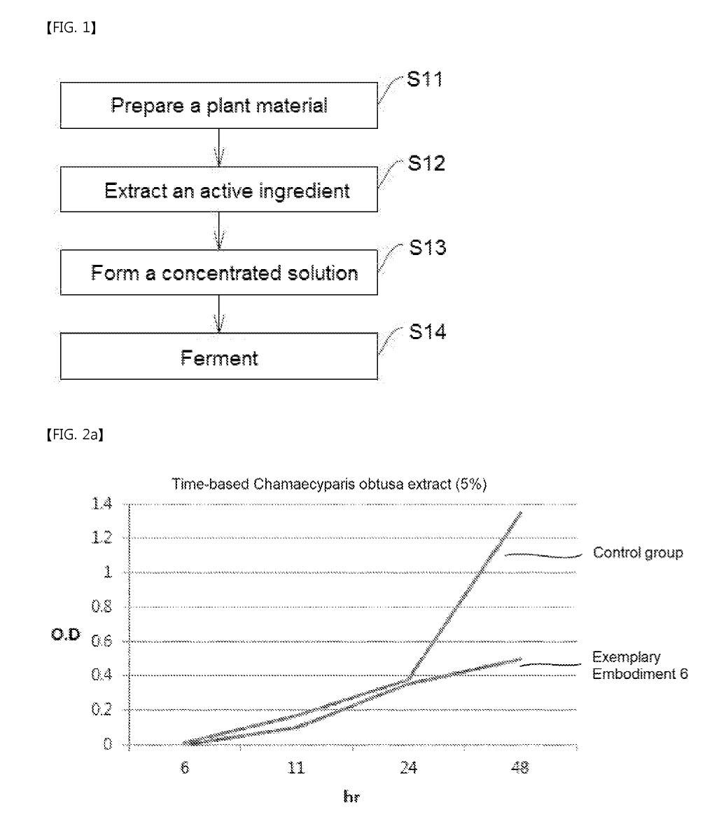

FIG. 1 illustrates a general view showing a process of producing a composition according to one exemplary embodiment of the present invention.

Referring to FIG. 1, the method for producing a composition according to the present invention may include a step of preparing a plant material (S11); a step of extracting an active ingredient from the plant material (S12); a step of forming a concentrated solution of the extracted active ingredient (S13); and a step of fermenting the concentrated solution (S14).

The plant material for producing the composition according to the present invention may corresponds to at least one selected from a group consisting of Taraxacum platycarpum, Centella asiatica, Ecklonia stolonifera, Bambuseae, Petasites japonicus, Chamaecyparis obtusa, Cactaceae and Aloe. Taraxacum platycarpum refers to a medical herb made of dehydrated (or dried) dandelion (or Taraxacum platycarpum Dahlst) of Compositae or plants belonging to the same family. Centella asiatica refers to a plant having its green leaf and stem used as medical ingredients and is used as a medical herb for treating broken skin, chronic ulcer, arthritis, and corniculate, and as a antipyretic. Herein, the Centella asiatica according to the present invention includes Centella asiatica that reseed in random regions. Ecklonia stolonifera refers to perennial marine algae abundantly containing minerals and belonging to Phaeophyta Laminariales laminariaceae. Bambuseae (or bamboo) corresponds to a plant belonging to gramineae (or poaceae), and the composition according to the present invention may include extracts of bamboo leaves. Petasites japonicas refers to a perennial herbaceous plant belonging to Compositae reseeding in humid areas near forests or valleys and the buds of this plant are used as oriental medical herbs, and the composition according to the present invention may mainly include extracts of Petasites japonicas leaves. Additionally, Chamaecyparis obtusa belongs to a needle-leaved evergreen arbor family and also refers to a plant that belongs to Thuja orientalis, which has poor low-temperature resistance and flame (or fire) resistance but high environmental tolerance. Chamaecyparis obtusa contains an anti-bacterial substance, which is known as Phytoncide, and which is well known for its effects of enhancing skin conditions including allergies, anti-bacteria, anti-stress, deodorizing and purifying effects, and increasing immunity, and the composition according to the present invention may mainly include extracts of Chamaecyparis obtusa leaves.

In order to produce the composition according to the present invention, each of the plant materials may be collected and its leaf, stem, or root may be prepared by being washed and cleaned, and, whenever required, each plant material may be prepared by being dried (or dehydrated) and ground (S11).

Although the prepared plant material may be extracted in accordance with an extracting method best-fitting each of the materials, considering the fact that the material should be applied to a human body, for example, methods such as hot-water extraction, ethanol extraction, microwave extraction, and supercritical extraction may be applied (S12). Thereafter, the extract may be formed into a concentrated solution (S13).

More specifically, the hot-water extraction, for example, may consist of a method of forming distilled water of 80 to 100 and soaking Chamaecyparis obtusa leaves for 2 to 6 hours. Thereafter, the extract may be filtered and processed with vacuum evaporation. The ethanol extraction may consist of a method of soaking Chamaecyparis obtusa leaves in ethanol and culturing for 2 to 5 days at 30 to 45, and filtering the extracted active ingredient and reducing pressure and concentrating. The microwave extraction may consist of a method of soaking Chamaecyparis obtusa leaves in distilled water for 10 to 50 minutes, processing the preparation with microwave, and filtering the extract and processing the filtered extract with vacuum evaporation. Finally, the supercritical extraction may correspond to a method of processing with carbon dioxide or pentane in 300 to 400 bar at a temperature of 70 to 90 for 2 to 6 hours and allowing the extraction efficiency to be 0.2 to 0.5 wt % of the sample mass weight.

According to the present invention, the concentrated solution may be directly applied as a composition for blocking ultra-violet (UV) rays, or the concentrated solution may be fermented (S14) and then applied as a composition for blocking UV rays.

For the fermentation, for example, bacteria such as Lactobacillus fermentum, Lactobacillus brevis, Lactobacillus plantarum, or Aspergillus niger may be used. Fermentation may be carried out for 2 to 6 days at a temperature ranging from 20 to 35, and the fermented solution may be centrifuged in a centrifuge for 10 to 30 minutes at 10,000 to 20,000 rpm. Thereafter, the centrifuged supernatant may be separated and then concentrated.

On the other hand, in order to gain the composition according to the present invention, solid fermentation may be applied. Solid fermentation may be created (or produced) in the form of powder by washing and drying (or dehydrating) a plant material, and by finely grinding the dehydrated ingredient. Thereafter, the powdered ingredient may be fermented for 2 to 6 weeks in a MRS culture medium (or medium) by using Lactobacillus bacteria, preferably Lactobacillus plantarum. The fermentation may be carried out, for example, at 20 to 35. Afterwards, the fermented substance may be dissolved in a solvent and may then be applied as a composition for a sunscreen (or sunblock).

The extract of the plant materials proposed above or the fermentation ingredient of the extract of the plant material may, for example, include materials, such as coumarinic acid, cinnamic acid, or ferulic acid, and may also include organic acid, sugar, Saponin, alkaloid, glycoside, and diverse forms of protein. Additionally, materials such as aciatic acid or asiaticoside may also be included.

### MODE FOR CARRYING OUT THE PRESENT INVENTION

Hereinafter, exemplary embodiments of the composition according to the present invention will be described.

### Exemplary Embodiments

### Exemplary Embodiment 1

Step 1: Taraxacum platycarpum leaves are collected, washed and dried, and then finely ground.

Step 2: The ground ingredient is soaked in distilled water of approximately 85 for approximately 3 hours and then filtered and processed with vacuum evaporation (approximately 4.57 wt % of the total weight).

Step 3: A sunscreen (or subblock) product is produced by mixing and agitating (or shaking) the following: Ingredient processed with vacuum evaporation 5.0 g, Propylene Glycol 10 g, Castor oil 1.0 g, Citric acid 0.2 g, and purified water 60 g.

### Exemplary Embodiment 2

Step 1: Centella asiatica is collected, washed and dried, and then finely ground.

Step 2: The ground ingredient is soaked in ethanol of approximately 40 for 2 days and then filtered, and the gained solution is processed with vacuum evaporation (approximately 4.54 wt % of the total weight).

Step 3: A sunscreen product is produced by mixing and agitating (or shaking) the following: Ingredient processed with vacuum evaporation 5.0 g, Propylene Glycol 10 g, Mineral oil 10 g, Citric acid 0.2 g, and purified water 60 g.

### Exemplary Embodiment 3

Step 1: Ecklonia stolonifera is collected, washed and dried, and then finely ground.

Step 2: The ground ingredient is put into in distilled water and processed with microwave at a temperature of approximately 60 for 30 minutes and then filtered, and the gained solution is processed with vacuum evaporation (approximately 4.21 wt % of the total weight).

Step 3: A sunscreen product is produced by mixing and agitating (or shaking) the following: Ingredient processed with vacuum evaporation 5.0 g, Propylene Glycol 10 g, Castor oil 1.0 g, Citric acid 0.2 g, and purified water 60 g.

### Exemplary Embodiment 4

Step 1: Bambuseae (or bamboo) leaves are collected, washed and dried, and then finely ground.

Step 2: The ground ingredient is processed with carbon dioxide at 350 bar at a temperature of 80 for 3 hours, and the extraction efficiency is set to 0.3 wt % of the total weight.

Step 3: A sunscreen product is produced by mixing and agitating (or shaking) the following: Ingredient processed with vacuum evaporation 5.0 g, Propylene Glycol 10 g, Mineral oil 10 g, Citric acid 0.2 g, and purified water 60 g.

### Exemplary Embodiment 5

Step 1: Petasites japonicas is collected, washed and dried, and then finely ground.

Step 2: The ground ingredient is processed by using the same method as Exemplary Embodiment 1, and the vacuum evaporation solution is gained, and then fermentation is carried out with Lactobacillus plantarum for 3 days at a temperature of 25, and then, after performing centrifuging, a concentrated solution is gained (approximately 2.41 wt % of the total weight).

Step 3: A sunscreen product is produced by mixing and agitating (or shaking) the following: Ingredient processed with vacuum evaporation 5.0 g, Propylene Glycol 10 g, Castor oil 1.0 g, Citric acid 0.2 g, and purified water 60 g.

### Exemplary Embodiment 6

Step 1: Chamaecyparis obtusa leaves are collected, washed and dried, and then finely ground.

Step 2: The ground ingredient is fermented in a MRS medium using Lactobacillus plantarum for 3 weeks at 25 and then a solid fermentation is gained. Then, after dissolving the solid fermentation in ethanol, the dissolved fermentation is centrifuged and filtered (approximately 3.25 wt % of the total weight).

Step 3: A sunscreen product is produced by mixing and agitating (or shaking) the following: Solid fermentation 5 g, Propylene Glycol 10 g, Mineral oil 10 g, Citric acid 0.2 g, and purified water 60 g.

### Exemplary Embodiment 7

Step 1: Cactaceae (or cactus) is collected, washed and dried, and then finely ground.

Step 2: The ground ingredient is soaked in ethanol of approximately 40 for 2 days and then filtered, and the gained solution is processed with vacuum evaporation (approximately 4.60 wt % of the total weight).

Step 3: A sunscreen product is produced by mixing and agitating (or shaking) the following: Ingredient processed with vacuum evaporation 5.0 g, Propylene Glycol 10 g, Mineral oil 10 g, Citric acid 0.2 g, and purified water 60 g.

### Exemplary Embodiment 8

Step 1: Aloe is collected, washed and dried, and then finely ground.

Step 2: The ground ingredient is put into in distilled water and processed with microwave at a temperature of approximately 60 for 30 minutes and then filtered, and the gained solution is processed with vacuum evaporation (approximately 4.81 wt % of the total weight).

Step 3: A sunscreen product is produced by mixing and agitating (or shaking) the following: Ingredient processed with vacuum evaporation 5.0 g, Propylene Glycol 10 g, Castor oil 1.0 g, Citric acid 0.2 g, and purified water 60 g.

Efficacy Test

1. Efficacy Test on Blood Flow Enhancement and Carboxy Therapy

The compositions according to the first to sixth exemplary embodiments have been treated on the calf and thigh areas of a human body for 20 days, and then blood samples were acquired after 10 days and 20 days of treatment in order to test the degree of blood flow enhancement. Thereafter, in order to perform an efficacy test for carboxy therapy, body fat, body water, fat free mass, and contour size of the thigh (thigh contour) were measured. Individuals determined as being in an obese state were selected as test subjects, and 25 test subjects were selected, and 20 test subject excluding 5 test subjects, who did not show any level of significance, were averaged.

The test results are shown below in the following table.

TABLE 1

| | Blood flow | | Body | | Body | | Fat | | Contour | |
|---|---|---|---|---|---|---|---|---|---|---|
| Emb. | enhancement | | fat | | water | | free mass | | size | |
| 1 | 4 | 5 | 0.3 | 0.3 | 0.4 | 0.5 | 0.5 | 0.6 | 0.6 | 0.7 |
| 2 | 3 | 4 | 0.2 | 0.4 | 0.3 | 0.3 | 0.4 | 0.5 | 0.6 | 0.8 |
| 3 | 4 | 5 | 0.3 | 0.8 | 0.4 | 0.6 | 0.6 | 0.8 | 0.5 | 0.6 |
| 4 | 5 | 6 | 0.3 | 0.6 | 0.3 | 0.6 | 0.7 | 0.9 | 0.7 | 0.7 |
| 5 | 5 | 5 | 0.7 | 0.8 | 0.5 | 0.4 | 0.6 | 0.8 | 0.6 | 0.8 |
| 6 | 12 | 16 | 1.6 | 2.2 | 1.5 | 2.0 | 2.4 | 2.9 | 1.9 | 2.0 |
| 7 | 5 | 4 | 0.8 | 0.7 | 0.6 | 0.4 | 0.5 | 0.4 | 0.7 | 0.8 |
| 8 | 5 | 4 | 0.6 | 0.8 | 0.6 | 0.5 | 0.5 | 0.4 | 0.6 | 0.7 |

* Blood flow enhancement was measured by taking blood samples for the calf area and by measuring the time consumed for saline solution to pass through by using a Micro channel array flow analyzer.
* Blood flow enhancement indicates the level of rate increase in %.
* Body fat, fat free mass, and contour size indicate the decreased amount, and body water indicates the increased amount, and each values are indicated in %.
* The body fat, body water, and fat free mass were measured by using a bio-electrical impedance analysis, and the contour size was directly measured.

2. Efficacy Test on Pityriasis and Tinea Pedis

Pityriasis (or Dandruff)

In order to carry out the efficacy test, Pityrosporum ovale was cultivated (or grown) for 27 to 72 hours in a culture medium (Pityrosporum medium) of MaltExtract 6%, Ox-bile 25, Tween 40 1%, Glycerol monoleate 0.25%. And, in order to cultivate (or grow) Trichophyton rubrum, firstly, a spore preparation has been prepared. Thereafter, bacteria cultivated in a solid medium are mixed in 0.5% NaCl 5 ml, and spreading is performed in the medium and then pushed by using a sliding glass. Subsequently, a vortex was formed in a falcon tube, and, then, the bacteria+NaCl solution was inserted in a beaker covered with a gauze, which was treated (or sterilized) in advance in an autoclave, so as to be filtered, thereby allowing only a liquid spore to remain. By using the above-described method, the strain, which has become the spore preparation, was cultivated in a SD (sabouraud dextrose) medium at 27 for 5 days.

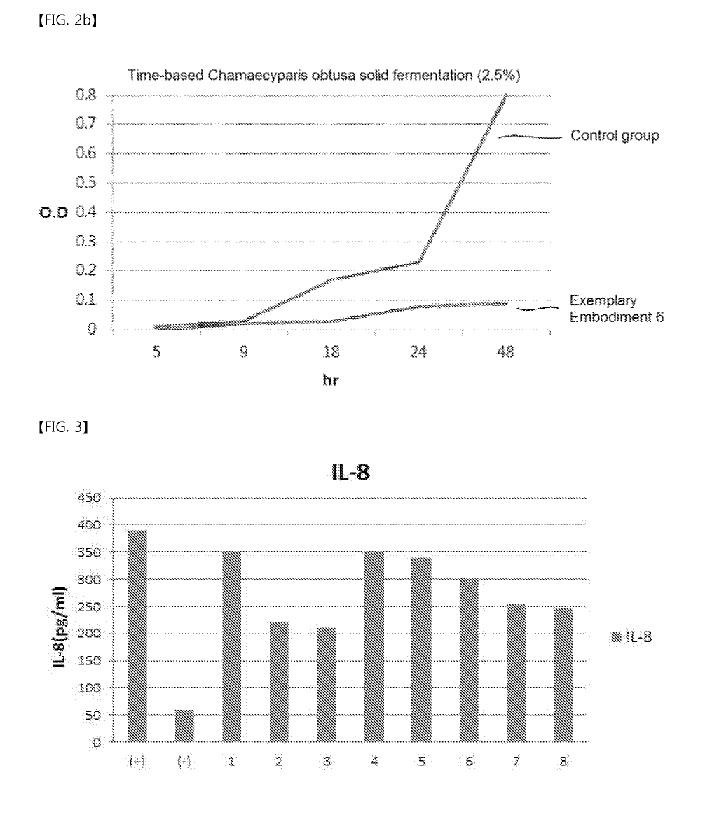

Test results of Chamaecyparis obtusa hot-water extraction extract and fermentation material on Pityrosporum ovale and Trichophyton rubrum are presented in FIG. 2a and FIG. 2b. Chamaecyparis obtusa was extracted by using hot-water extraction and was fermented by using L. platarum. Referring to FIG. 2a and FIG. 2b, it can be known that the fermentation material of Chamaecyparis obtusa has a high anti-bacterial effect on Pityrosporum ovale and Trichophyton rubrum.

3. Sun Protection Factor Test

Sun Protection Factor Index (SPF Index) and safety evaluation has been carried out on the sunscreen products obtained from exemplary embodiments 1 to 8. For the concentration, the sunscreen products obtained from exemplary embodiments 1 to 8 were used as the standard sample, and the concentration was adjusted by additionally including extracts to each sample.

The sunscreen product was applied to 100 test subjects at a 24-hour interval and cumulative stimulus was guided for 20 days. For the control group, Zinc oxide and Titanium dioxide, which correspond to inorganic-type sunscreen products, were used.

UV Spectrophotometer and a Xenon Arc Lamp have been used for the measurement, and the measurement wavelength ranges from 290 to 400 nm, and the SPF has been measured 15 minutes after applying the sunscreen product.

The results for the sun protection factor (SPF) index test are as shown below.

TABLE 2

| | 1 | | 2 | | 4 | |
|---|---|---|---|---|---|---|
| | UVB | UVA | UVB | UVA | UVB | UVA |
| Emb. 1 | 13.04 | 2.24 | 16.08 | 10.57 | 20.20 | 18.25 |
| Emb. 2 | 14.08 | 3.25 | 17.25 | 14.21 | 20.10 | 18.21 |
| Emb. 3 | 13.28 | 2.75 | 16.76 | 12.92 | 21.45 | 19.30 |
| Emb. 4 | 15.24 | 2.84 | 17.16 | 10.27 | 22.43 | 16.27 |
| Emb. 5 | 15.65 | 3.84 | 16.72 | 12.12 | 22.24 | 19.24 |
| Emb. 6 | 16.12 | 3.92 | 18.24 | 14.25 | 22.48 | 20.15 |
| Emb. 7 | 17.24 | 4.52 | 20.15 | 18.92 | 24.22 | 23.34 |
| Emb. 8 | 17.09 | 4.31 | 19.84 | 18.27 | 23.31 | 23.01 |
| ZO | | 5.42 | | 15.72 | | 42.83 |
| TD | | 8.18 | | 24.42 | | 50.12 |

1 represents the standard sample, and 2 and 4 respectively signify the standard sample having its concentration increased to 2 times and 4 times.

Repeated epidermal reaction was tested in each test, and evaluation was made by categorizing the results to Levels 0 to 4 (No visible reaction, Mild erythema, Intense erythema, Intense erythema with edema, Intense erythema with edema & vesicle), and tendencies in epidermal reaction have been recorded in accordance with skin color tone level, dryness level, hydration (or moisture) level, and laser blood flow rate. There has been no test subject showing a reaction equivalent to Level 1 or higher.

On the other hand, UVA was irradiated, and Phototoxic indexes were calculated after a 24-hour period and a 48-hour period. Herein, the Phototoxic index is indicated as Phototoxic index=irradiation index of UV-irradiated area and irradiation index of UV non-irradiated area.

The Phototoxic index may be indicated in values ranging from 0 to 8.0, wherein 0 represents Non Phototoxic, and wherein 8 indicates Extremely phototoxic. Among the 100 test subjects 20 subjects showed a Phototoxic index of approximately 0.3 during the primary testing.

On the other hand, in order to test the pharmaceutical characteristics of the composition according to the present invention, each of the compositions produced in accordance with exemplary embodiments 1 to 8 was stimulated with LPS (lipopolysaccharide) in a HaCa T cell line, and, accordingly, a change in the amount of cytokine was measured. 0.1/of each composition was used, and a change in the cytokine was detected by measuring the amount of interleukin-8 (IL-8), and the corresponding results are presented in FIG. 3.

In FIG. 3, + represents +LPS, and − represents −LPS, and 1 to 8 represent the respective exemplary embodiment. As it can be known from FIG. 3, it can be known that the amount of cytokine is reduced due to the composition according to the first to eighth exemplary embodiments, and it can also be known that the substance according to the present invention has an anti-infection effect or pharmaceutical effects.

As it can be known from the test examples presented herein, it can be known that the composition according to the present invention shows the same effects as an inorganic-type UVB sunscreen product and an organic-type UVA sunscreen product without causing any epidermal (or skin) irritation. Therefore, the composition according to the present invention is advantageous in that it can be used as a replacement for such products.

The composition according to the present invention is advantageous in that the composition is produced from a plant material, and that the composition does not cause any side effects, such as infections (or skin trouble). The composition according to the present invention is also advantageous in that, since the composition correspond to a natural ingredient, the composition may be added and used in diverse natural functional cosmetics ingredient. Furthermore, the composition according to the present invention is also advantageous in that the composition has an excellent texture, and that the composition can be easily removed from the skin.

Although the present invention has been described above in detail with reference to the exemplary embodiments set forth herein, anyone skilled in the art may devise diverse variations and modifications of the present invention without departing from the technical scope and spirit of the present invention by referring to the exemplary embodiments presented herein. It should be understood that the present invention will not be limited by such varied and modified inventions. Nevertheless, the present invention shall be limited to the scope and spirit of the appended claims.

### INDUSTRIAL APPLICABILITY

The composition according to the present invention may be used for producing functional cosmetics.

What is claimed is:

1. A composition for producing functional cosmetics, comprising:
at least one selected from the group consisting of Taraxacum platycarpum, Centella asiatica, Ecklonia stolonifera, Bambuseae, Petasites japonicus, Chamaecyparis obtusa, Cactaceae and Aloe and comprises an extract which has been extracted from the at least one by means of hot-water extraction, ethanol extraction, microwave extraction or supercritical extraction, or a fermentation ingredient of the extract, wherein the fermentation ingredient has been fermented using Lactobacillus.

2. The composition of claim 1, wherein the cosmetics corresponds to functional cosmetics for enhancing blood flow, treating acne, generating carboxy, treating pityriasis (or dandruff), treating tinea pedis (or Athlete's foot), blocking UV rays, or relieving epidermal infections.

3. The composition of claim 1, wherein the extract or fermentation ingredient comprises at least one selected from a group consisting of coumarinic acid, cinnamic acid, ferulic acid, aciatic acid, and asiaticoside.

4. A method for producing a composition for producing function cosmetics using a plant material extract or a fermentation material thereof, comprising the steps of:
preparing at least one plant material selected from a group consisting of Taraxacum platycarpum, Centella asiatica, Ecklonia stolonifera, Bambuseae, Petasites japonicus, Chamaecyparis obtusa, Cactaceae and Aloe;
forming an extract by processing the at least one plant material by means of hot-water extraction, ethanol extraction, microwave extraction or supercritical extraction; and
forming a concentrated solution of the extract.

5. The method of claim 4, further comprising a step of:
fermenting the extract by using Lactobacillus.
